(19) United States
(12) Patent Application Publication
(10) Pub. No.: US 2025/0009266 A1
(43) Pub. Date: Jan. 9, 2025

(54) BLOOD SAMPLE COLLECTION DEVICE WITH TIME STAMP AND SIMULTANEOUS ENVIRONMENTAL SAMPLE

(71) Applicant: Nightingale Health Oyj, Helsinki (FI)

(72) Inventor: Brandon T. Johnson, Cambridge, MA (US)

(21) Appl. No.: 18/891,384

(22) Filed: Sep. 20, 2024

Related U.S. Application Data
(63) Continuation of application No. 16/657,069, filed on Oct. 18, 2019, now abandoned.
(60) Provisional application No. 62/749,160, filed on Oct. 23, 2018.

(51) Int. Cl. A61B 5/15; B01L 3/00; G16H 10/40

(52) U.S. Cl. CPC A61B 5/150343; A61B 5/150755; B01L 3/508; G16H 10/40; A61B 2560/0252; A61B 2562/029; B01L 2300/025; B01L 2300/045; B01L 2300/047; B01L 2300/0663

(57) ABSTRACT

A blood sample collection device includes a two-piece housing that encompasses a port at which a fingertip blood sample is collected. After the sample is taken, the two-piece housing is moved to a closed position to protect and optionally process the sample. A sample event detection circuit triggers a recording of a time when the sample was taken. The sample event detection circuit may determine when the housing is closed, or may detect the presence of blood in or near the sample port. The electronics may optionally record environmental conditions such as temperature or humidity, etc. at the time the sample is taken. The recorded data may then be used to enhance the value or accuracy of lab test, or for other purposes, such as inventory management.

### CROSS REFERENCE TO RELATED APPLICATIONS

This application is a continuation of U.S. Nonprovisional application Ser. No. 16/657,069, filed Oct. 18, 2019, which claims priority to U.S. Provisional Patent Application entitled “Blood Sample Collection Device with Time Stamp and Simultaneous Environmental Sample”, Ser. No. 62/749,160, filed Oct. 23, 2018, the entire contents of which are hereby incorporated by reference in their entireties.

### BACKGROUND

### Technical Field

This patent relates to devices and methods for blood sample collection.

### Background Information

Blood used for diagnostic testing is most often extracted from a patient with a hypodermic needle and collected in a test tube. The collected blood is then packaged for shipment to a remote lab where various diagnostic tests are performed. However, many diagnostic tests require significantly less volume than the actual collected sample. Separation of cellular components from the sample is also needed for some tests.

Many tests only require small blood samples, where a finger stick rather than a hypodermic needle can produce enough blood. But this small amount of blood cannot be easily transported to a remote lab. If the testing method cannot be immediately used at the same time the blood is extracted, convenient and reliable methods of collecting, prepping, and preserving small amounts of blood are still needed.

US Patent Publication US2014/0050620A1, assigned to Boston Microfluidics, Inc., describes several ways to implement a portable, user-friendly device for collecting a biological fluid sample and stabilizing it for transport to a remote lab. The devices include a small, hand-held housing that provides a chamber for collecting a fluid sample. Movement of the housing itself, and/or mechanisms located within the housing, initiate collection of a predetermined, metered volume of a fluid sample. The devices may also stabilize the collected sample and/or seal the sample in the chamber. Other mechanisms in the device may mix the collected sample with a reagent.

### SUMMARY

These small hand-held devices are very convenient for collecting a blood sample at a remote location. However, what is needed is a convenient way to accurately measure and record when the sample was collected. It would also be helpful to record other things, such as environmental conditions, before, during, and after the sample is taken.

In one approach to solving this problem, a fluid sample collection device includes sensors that record the time and/or conditions when the sample was taken. The sensors, which may be electronics or other passive non-electronic sensors (such as a paper that changes color or in some other visual way) may optionally record time or indicate extremes of environmental conditions such as temperature, humidity, etc. The time and/or other sensor recordings are triggered such as when the user closes the device, or when blood is detected entering into the device.

The data recorded at the time of taking a sample may then be used to enhance the value or accuracy of lab tests or determine that the sample is valid or invalid.

However, the recorded data may also be used for other purposes, such as inventory management. For example, the device itself can now be used to determine if the blood sample may have been taken beyond an expected shelf life for the device.

In other configurations, the electronics or other sensors may be enabled to continuously monitor the time and environmental conditions from a point of manufacture, until the time it is disassembled to retrieve the blood sample. This information can then be made available to the user or laboratory, to ensure that expired devices are not used, or if the sample has been exposed to extreme environmental conditions while in transit to the laboratory facility.

### BRIEF DESCRIPTION OF THE DRAWINGS

FIG. 1 is an isometric view of a blood sample collection device in the open position, before it is used.

FIG. 2 is a view of the collection device in the closed position.

FIG. 3 is an exploded view showing components of one example of the collection device.

FIG. 4 is a block diagram of example electronics located inside the device.

FIG. 5 is a process flow diagram.

FIG. 6 is a view of the collection device placed in a container, such as bag, for shipping to or from a point of use.

### DETAILED DESCRIPTION OF PREFERRED EMBODIMENTS

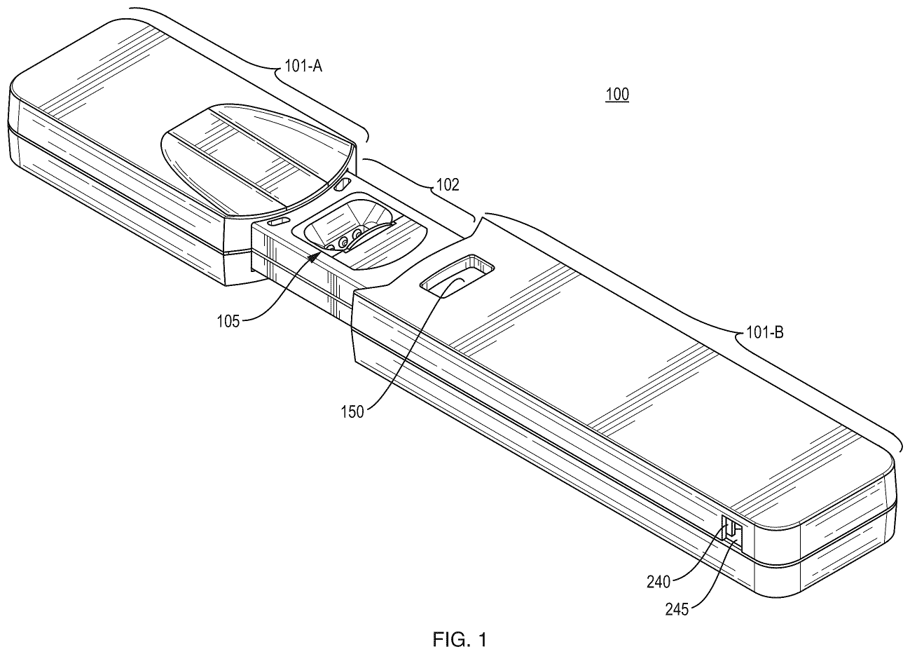

FIG. 1 is an isometric view of an example blood collection device 100 that includes a two-piece housing 101. The housing 101 includes a first housing piece 101-A and second housing piece 101-B. In this view, the housing is in the open position with the two pieces 101-A, 101-B spaced apart from one another, to provide access to a sample port 102. One or more windows 150 in the housing permits a user to confirm the status of one or more portions of a blood sample stored therein. The windows 150 may also provide a view of an electronic display within the device 100.

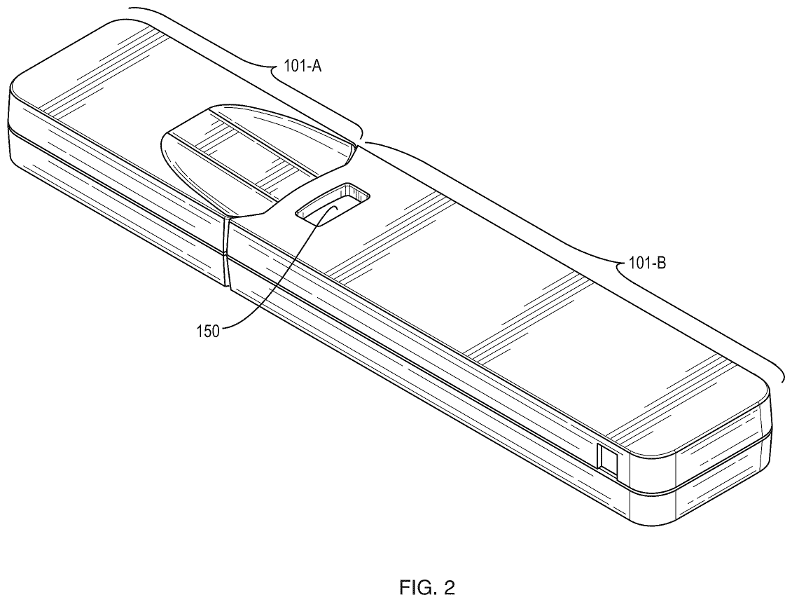

FIG. 2 is a similar isometric view of the device 100. In this view, a blood sample has been taken via the sample port 102, and the two housing pieces 101-A and 101-B have been pushed together to place the device 100 in a close position. In this closed position, the window 150 still provides access to the blood collection status, and or information shown on an electronic display.

The device 100 is typically used as follows. The device 100 is initially presented in its open position, to provide access to the sample port 102. A user, such as a patient herself or a health care professional, then uses a lancet to produce a blood sample such as from a finger tip. Drops of whole blood are then taken with the finger positioned near to, above, adjacent to, or even in contact with the sample port 102 to minimize blood spillage. Blood then flows from the sample port 102, introducing whole blood into the rest of the device 101. As will be explained in more detail below for one embodiment, capillary action may cause blood to flow from the sample port 102 into one or more collection capillaries 105 adjacent the sample port. The capillaries 105 can optionally be pre-coated with reagents such as heparin and/or EDTA for subsequent stabilization and preservation of the sample. The collection capillaries 105 can have a known and predetermined volume, in which case the incoming sample is precisely metered. The collection capillaries 105 then direct the metered sample to a media inside the device housing 101.

The user, who can be the patient himself/herself or a healthcare professional, then manually closes the device 100 by pushing the two housing pieces 101-A, 101-B together. The motion associated with closing the housing may then optionally enact one or more microfluidic mechanisms that further process the sample now securely stored inside the device 100.

As will be explained in more detail below, in one embodiment, the act of closing the housing 101 is detected by the device. This closure event then triggers recording of certain data, such as time, environmental conditions, or other information.

The window 150 may include a transparent piece of material that enables the user to view the sample port 102 and/or collection capillaries 105. In that way, an indication of whether a sufficient sample of blood is being drawn into the device 100 (when the housing 101 is in the open position) or was drawn into the device (when the housing 101 is in the closed position).

In other configurations, the same or another window 150 may include a view of an electronic display that permits the user to view information collected by the device 100.

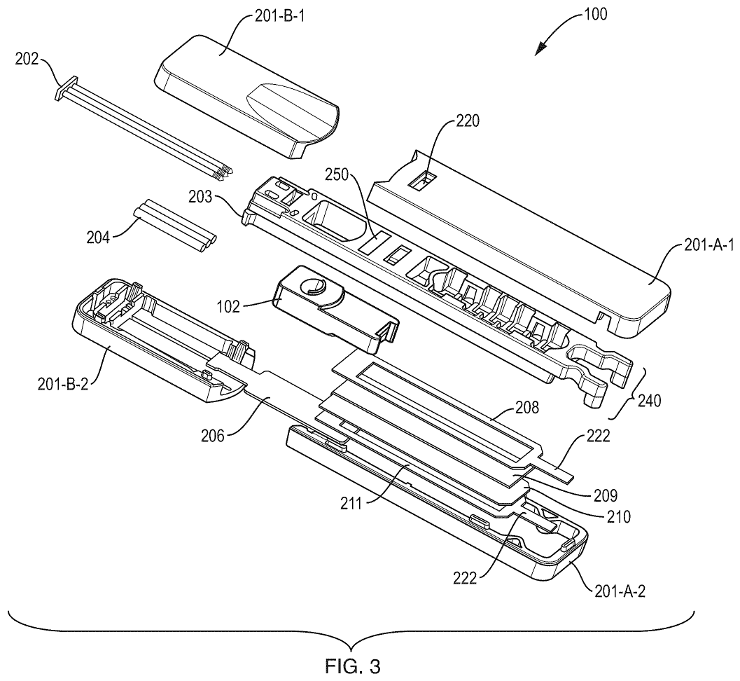

FIG. 3 is a more detailed view of the components of the device 100. The first housing piece 101-A consists of a top case 201-A-1 and bottom case 201-A-2, and second housing piece 101-B consists of a top case 201-B-1 and bottom case 201-B-2.

A backbone structure 203 supports sensors such as electronics 250 and other components. For example, a plunger rack 202 is also supported by the backbone structure 203. The backbone structure 203 may further include a ribbed section to support a desiccant tablet (not shown) to further dry the collected sample. The backbone structure 203 may also provide a ratcheting housing closure mechanism 240, which is activated when the two housing pieces 101-A, 101-B are pushed together.

Metering capillaries 204 engage the sample collection port 102 which may be further defined by a silicone inlay structure shaped to fit a hole 221 in backbone 203. The capillaries 204 can optionally be pre-coated with reagents, heparin, EDTA, or other substances.

In one arrangement, the plunger rack 202 firmly engages with the capillaries 204, creating a shutoff that blocks off any excess sample while also pushing the metered sample volume to the subsequent downstream processing steps.

A base 206 that fits into the backbone 203 also provides mechanical support for a blood collection membrane which may consist of a sample media 209. The sample media 209 may be further supported and/or held in place by other components that assist with handling the sample media 209, such as when it is removed from the device 101 for processing by a lab. These may include a top frame 208, mylar support 210, and bottom frame 211. The top 208 and bottom 211 frames may have extensions or tabs 222 on an outboard end. The tabs 222 further assist with handling the media once it is removed from the housing 101.

The sample media 209 may be a Pall membrane (sold by Pall Corporation), an LF1 glass fiber membrane (sold by General Electric Company) or some other media designed to receive serum or whole blood which it then separates into a blood portion and a plasma portion. A media such as LF1 paper has a fibrous structure that causes differential migration of the sample, with a slower rate for red cells, resulting in a gradual separation of plasma sample as it migrates down the paper. The membrane 209 can optionally be previously impregnated with heparin, EDTA, sugars, or other stabilization agents.

It can now be appreciated that the action of closing the housing pieces together causes the sample to exit the capillaries and be deposited onto the sample media 209. In particular, each of the plungers 202 is aligned with a corresponding one of the capillary tubes 204. The capillary tubes 204 are in turn held in place within the silicone rubber inlay. As the housing sections are closed together, the plungers 202 are forced into the capillary tubes 204, which in turn force blood towards a slot in the collection element.

If an inlay is used to define the sample port 102 it should have an elasticity that is sufficient to hold the capillary tubes 204 in place while the plungers 202 are forced into them. The elasticity of inlay 102 may also be chosen to seal the space around the capillary tubes and the inlay to prevent blood from flowing around the capillary tubes.

The closed housing also creates a small and isolated internal air space above the sample media 209. The sample can be further encouraged to dry with the aid of one or more desiccant tablets (not shown) supported by the backbone 203 adjacent where the sample media 209 sits when the housing is in the closed position.

During or after the housing is closed, a ratcheting mechanism provided by tines 240 on the end of the backbone 203 encourage the housing to remain shut. For example, the tines 240 may act as a ratcheting pall and engage small holes 245 in the end of housing piece 101-A when the housing is pushed shut. The tines 240 may be shaped to permit opening of the housing only with a special pinching tool that accesses small holes 245 in the side of the housing to releases the ratchet pawl. Thus, once the device 100 is closed by pushing the housing pieces 101-A, 101-B together, the blood sample remains enclosed within, and ready for transport to a remote lab.

More details of the internal components of one example device 100, including the plunger, capillary tubes, silicone inlay, ratcheting pall and other features are found in our co-pending U.S. Provisional Patent Application Ser. No. 63/577,761 filed Oct. 27, 2017 entitled “Blood Metering and Storage Device”, the entire contents of which are hereby incorporated by reference.

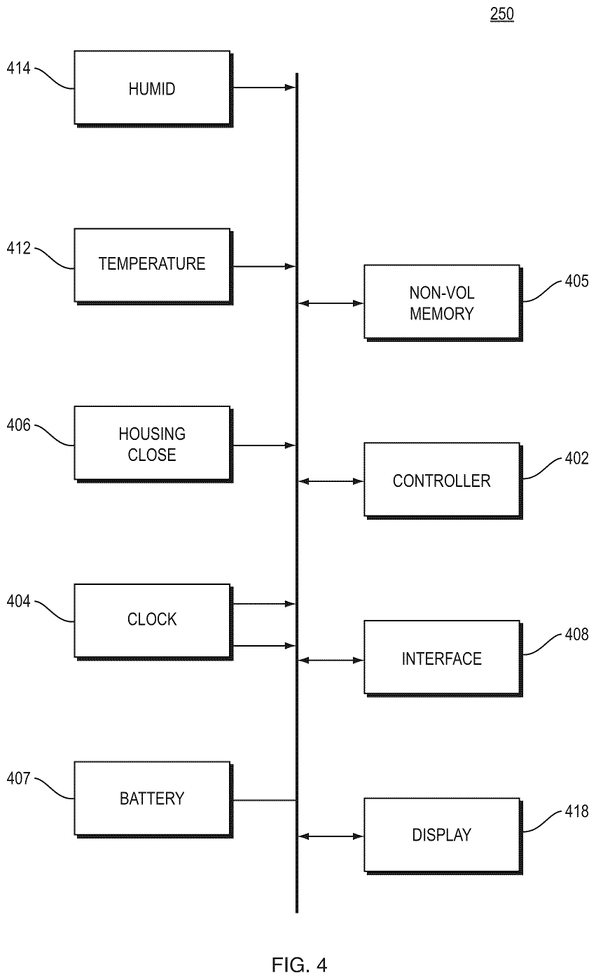

In one embodiment, shown in FIG. 4, a blood sample event sensor 406 detects closure of the housing is detected by electronics 250. The electronics 250 (which may be located in the backbone 203) may include at least a controller 402, a clock 404, a memory 405, a blood sample event sensor 406, interface 408, and power source such as a battery 407. Optional features may include other sensors such as a temperature sensor 412, humidity sensor 414, and other environmental sensors (not shown). An optional display 418 may be included in a position to be visible external to the device 100, such as beneath a window 220. or on some other location of one of the housings

The controller 402 may be a hard-wired fixed logic circuit such as a custom semiconductor integrated circuit, or one or more programmable logic circuits, such as an application-specific integrated circuit (ASIC) or field programmable gate array (FPGA). The controller 402 may also be a programmable device such as a microcontroller or microprocessor. The memory 405 may be a hard-wired fixed logic circuit such as a set of registers, or a Random Access Memory (RAM) such as a non-volatile memory (NVRAM), or other electronics that can retain data if power is lost or when the battery 407 is switched off.

In one configuration, the blood sample event sensor 406 determines when the housing 100 is placed in the closed position. Such a sensor thus provides a signal to the controller 402 when the first 101-A and second 101-B pieces of the housing are pushed together. This type of sensor 406 may use mechanical, proximity, magnetic, light, or other technologies for determining a relative position of the two housing pieces.

In other configurations, rather than detecting when the housing is closed, the sensor 406 may instead detect the presence of blood having been introduced into the port 102. A blood presence sensor 406 may detect a change in a light level indicating the housing has been closed, or an optical wavelength unique to blood. The sensor 406 may also be magnetic (e.g., detecting the presence of ferrous component such as iron in the blood), or a fluid or moisture sensing, or motion in a capillary, to determine when blood is introduced into the device 100. The sensor 406 may be triggered by any small amount of blood, such as even a single drop, or when a more substantial amount of blood has been introduced, such as when the sample well is filled to a certain predetermined level sufficient for the tests being applied.

A combination of housing position and blood presence sensors 406 may also be used.

The clock 404 provides data such as time of day and/or a date to the controller 402.

The interface 408 provides a way to extract data from the electronics. The interface may be wired or wireless. In the implementation shown, the interface is for example, a wireless Bluetooth interface that eliminates the need for an external physical connector on the device 100. However, a wired interface may also be provided, such as Universal Serial Bus (USB), where the connector can be placed somewhere on the housing 100.

The display 418 provides another way for the controller to present data and status information to a user.

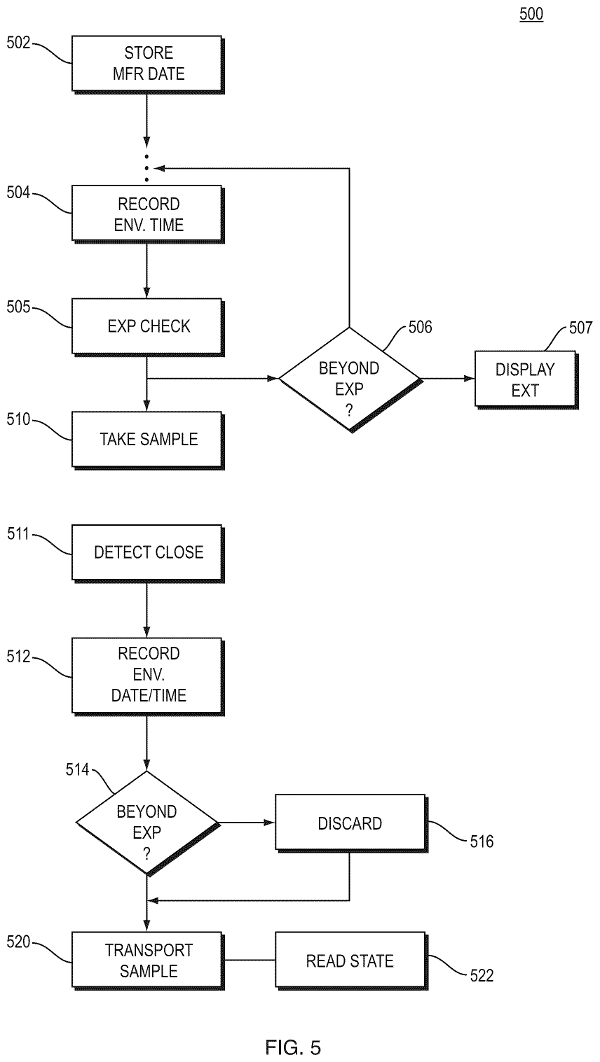

FIG. 5 is an example process flow 504 using the device 100 with the blood sample detector 406. In this example, in a first step 502, a date and time of manufacture are stored by the controller. The device 100 is then shipped to a warehouse or storage facility awaiting use. Periodically, the controller 402 enter state 504 to record a current date and time. In state 505, current environmental conditions may also be recorded. If the device has been in storage longer than a predetermined shelf life in state 506, or some environmental condition has been exceeded (such as extreme temperature and/or humidity over some predetermined time), then in 507 an indication can be provided on the display that the device has expired. That indication can be made via the electronic display. Otherwise processing can return back to 504 where environment and time are checked again.

As mentioned previously, passive, non-electronic (chemical) sensors may be used in place of some or all of the electronics. These passive sensors may include a treated substrate included within the housing that changes color when exposed to temperature (or humidity) extremes or over time. Other chemical sensors, such as the visually changing paper (VCP) described in U.S. Pat. No. 6,452,873 can be included within the housing to indicate an elapsed or expiry time. Still other chemical sensors can be triggered by lack of light exposure, such as when the housing is closed.

Eventually the device is brought to a point of use where a blood sample will be taken. Before taking a blood sample, the user or health care professional may interact via the interface 408 and/or display to determine whether device is expired or not.

In any event, in state 510 a blood sample is introduced into the port 102 and the device processes and stores the sample as explained above.

State 511 is entered when the blood sample event is detected, such as via a housing closure sensor 406. In state 512 the current date and/or time again recorded. Because the recording is automatically triggered at or near the actual time at which the sample was taken, an accurate indication of the time when the sample was taken is assured. If in state 514 the device 100 also happens to be beyond its expiration date when the sample was taken, then this may also be recorded in state 516.

In state 520 the device 100 is then placed in transit to a laboratory. After arrival at the lab, the storage media 210 is extracted from the device (such as by prying open the housing 101) and recorded information is read in state 522. Lab personnel may now determine a time when the sample was taken and whether or not the device 100 was in transit too long, exposed to extreme conditions either when in initial storage or during transit, or if the device was in an expired state at the time the sample was taken.

It should be understood that not all of the steps shown in FIG. 5 need to be carried out. For example, an important step is step 510 to record the time and date of the sample when the housing is closed. However, in some implementations, continuous monitoring of environmental conditions, or monitoring conditions at the time of closure and/or while devices in inventory or in transit may not be necessary.

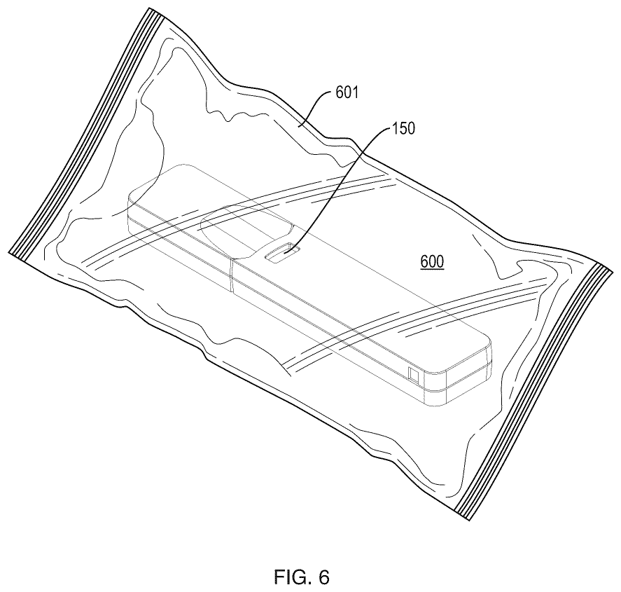

FIG. 6 illustrates another use of a sensor, to detect when the device 600 has been removed from a container 601 such as a sealed bag in which it is shipped. Here the sensor may be placed on or in the device, such as beneath window 150) to react to exposure to light when the device is removed from the bag. Note that the device 600 is partially visible in FIG. 6 only for the sake of illustration, and in fact the bag 601 would likely be opaque to prevent light from reaching the sensor until the device is removed from the bag 601. The sensor may trigger an event such as starting a timer, or other events, analogous to the previously described housing closure event.

It should be understood that in light of the above, various modifications and additions may be made to the device without departing from the true scope of the inventions made.

1. A blood sample collection device comprising:
a housing having an open position and a closed position;
a sample collection port, accessible for collecting fluid with the housing in the open position;
a sample event sensor, for detecting when a blood sample has been introduced into the device.

2. The device of claim 1 wherein the sample event sensor is a housing event closure detection circuit.

3. The device of claim 1 wherein the sample event sensor detects when a predetermined amount of the blood sample is collected.

4. The device of claim 1 additionally comprising:
a state recording circuit, triggered by the sample event sensor, for recording at least one state of the device.

5. The device of claim 4 where the recorded state is one or more of a time, or an environmental condition including one or more of temperature or humidity.

6. The device of claim 2 where the detection circuit is one or more of a light, magnetic, fluid, or motion sensor for detecting the presence of blood introduced adjacent the sample port.

7. The device of claim 1 where the sample event sensor detects when the housing is moved from the open position to the closed position.

8. The device of claim 7 where the sample event sensor is one or more of a mechanical, light, or proximity sensor.

9. The device of claim 1 additionally comprising:
a sample storage media disposed within the housing, for storing blood collected via the sample port.

10. The device of claim 9 additionally comprising:
a mechanically actuated fluid controller, configured to dispense blood from the sample collection port onto the sample storage media when the housing is moved from the open to the closed position.

11. The device of claim 1 additionally comprising:
an assay for processing the blood sample collected via the sample port.

12. The device of claim 1 additionally comprising:
a visual indicator triggered by the sample event sensor, comprising one or more of a temperature, time or humidity sensitive paper, or an electronic indicator.

13. A method of collecting a blood sample comprising:
providing a sample collection device having a housing and a sample port exposed when the housing is in an open position;
detecting when the sample port has been used to introduce blood into the device;
recording, within the sample collection device, a time at which blood was introduced into the device; and
moving the housing to a closed position.

14. The method of claim 13 wherein
the step of recording records a time at which the housing was moved to the closed position.

15. The method of claim 13 wherein
the step of recording records a time at which a predetermined amount of blood passes through the sample port.

16. The method of claim 13 additionally comprising:
during the step of moving the housing to a closed position, further processing the blood sample.

17. The method of claim 13 wherein further processing the blood sample additionally comprises one or more of:
directing the sample to a storage media, drying the sample, treating the sample with a reagent, assaying the sample, or separating plasma from the sample.

18. A blood sample collection device comprising:
a housing having an open position and a closed position;
a sample collection port, accessible for collecting fluid with the housing in the open position;
an event sensor, for detecting when the device is removed from a container.
